(19) United States
(12) Patent Application Publication
(10) Pub. No.: US 2023/0416841 A1
(43) Pub. Date: Dec. 28, 2023

(54) INFERRING TRANSCRIPTION FACTOR ACTIVITY FROM DNA METHYLATION AND ITS APPLICATION AS A BIOMARKER

(71) Applicant: Fred Hutchinson Cancer Center, Seattle, WA (US)

(72) Inventors: Michael Haffner, Seattle, WA (US); Brian Hanratty, Seattle, WA (US); Mohamed Adil, Redmond, WA (US); Gavin Ha, Seattle, WA (US)

(73) Assignee: Fred Hutchinson Cancer Center, Seattle, WA (US)

(21) Appl. No.: 18/340,699

(22) Filed: Jun. 23, 2023

Related U.S. Application Data
(60) Provisional application No. 63/355,484, filed on Jun. 24, 2022.

(51) Int. Cl. C12Q 1/6886; C12Q 1/6869; C12Q 1/6806

(52) U.S. Cl. CPC C12Q 1/6886; C12Q 1/6806; C12Q 2600/154; C12Q 1/6869

(57) ABSTRACT

A method of treating prostate cancer based on determining genome-wide methylation profile at a plurality of transcription factor binding sites in the total DNA obtained from cell-free biological samples from a subject suffering from prostate cancer are provided herein. Also described are methods for determining suitable treatment regimens for prostate cancer and methods for treating prostate cancer patients, based around selection of the patients according to the methods of the disclosure. The disclosure also relates to computer-implemented methods for identifying, diagnosing, staging, or otherwise characterizing cancers, in particular advanced prostate cancer. The methods of the present disclosure relate, inter alia, to isolating and analyzing the human DNA component from cell-free samples.

### CROSS-REFERENCE TO RELATED APPLICATION

This application claims the benefit of U.S. Provisional Application No. 63/355,484, filed Jun. 24, 2022, the disclosure of which is incorporated herein by reference in its entirety.

### BACKGROUND

Prostate cancer (PC) is the most prevalent non-skin malignancy diagnosed in men within the Western world, causing over 35,000 annual deaths in the United States alone. The clinical presentation of PC is diverse, ranging from localized indolent cases to rapidly progressing lethal metastatic diseases. PC is widely known to be an androgen-dependent tumor, with the androgen receptor (AR) playing a pivotal role in disease progression.

Despite notable therapeutic advancements in recent years, metastatic PC remains incurable and poses a significant healthcare and societal burden. The standard therapy for metastatic PC encompasses various pharmacological approaches that target the AR signaling axis. Although initial responses to AR-targeted therapies can be substantial, most patients eventually develop resistance. About 20-30% of advanced tumors which are resistant to standard therapies exhibit histologic and molecular characteristics that are divergent from conventional prostatic adenocarcinoma. In particular, tumors to lose AR expression and gain NE (neuroendocrine) features (termed neuroendocrine prostate cancers, NEPC) show a highly aggressive clinical course and due to the absence of robust biomarkers they are often detected too late and opportunities for alternative therapies are missed. A definitive diagnosis of NEPC can only be obtained through biopsies of metastatic lesions which are highly invasive and do not allow comprehensive sampling of the entire tumor burden. Therefore, there is an urgent need to identify novel biomarkers that can detect NEPC in a minimally invasive fashion for diagnosis providing for better therapeutic interventions.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides methods and systems for assessing (e.g., modeling) genome-wide methylation patterns at transcription factor (TF) binding sites (TFBSs) and using this information to detect, assess, diagnose, treat, and analyze disease states and identify treatment responsiveness. Cancer cells exhibit drastically different DNA methylation patterns at TFBSs than normal cells. Therefore, by identifying genome-wide methylation patterns at TFBSs in cancer cells, it is possible to distinguish cancer cells from normal cells. Next-generation sequencing-based genome-wide assays may be used to provide methylation status at TF-binding sites (TFBSs) in total DNA obtained from a subject. Circulating nucleic acid molecules from liquid biopsies, such as cell-free DNA (cfDNA), may provide an easily accessible source of nucleic acid (NA) for determining genome-wide methylation patterns at TFBSs.

The present disclosure provides methods and systems for assessing genome-wide methylation patterns from cfDNA or total DNA obtained from cell-free biological samples of a subject to provide information about TFs for applications relating to disease identification, prediction, staging, treating, and/or identifying treatment responsiveness. Methods and systems are described herein for using methylation pattern at TFBSs determined from DNA obtained from a biological sample of a subject (e.g., cfDNA). In some examples, the information may be used as inputs into machine learning models useful in many of these applications such as disease identification, prediction, staging, treating, and identifying treatment responsiveness.

In an embodiment, the present disclosure provides a method of treatment of prostate cancer. In some embodiment, the method comprises determining a molecular phenotype of the prostate cancer. In some embodiments, the method comprises administering to the subject an effective amount of at least one therapeutic agent based on the determination of the molecular phenotype of the prostate cancer.

In a related embodiment, the present disclosure also provides a method of determining the molecular phenotype of the prostate cancer. In some embodiments, the method comprises obtaining a biological sample from a subject suffering from prostate cancer. In some embodiments, the biological sample comprises a cell-free biological sample. In some embodiments, the method comprises isolating/obtaining DNA from the biological sample of the subject suffering from prostate cancer. In some embodiments, the method comprises isolating/obtaining total DNA from a cell-free biological sample of the subject suffering from prostate cancer. In some embodiments, the method comprises determining genome-wide methylation profile at a plurality of transcription factor binding sites in the total DNA obtained from the cell-free biological sample of the subject suffering from prostate cancer to obtain genome-wide methylation sequence reads at each of the plurality of transcription factor binding sites. In some embodiments, the method further comprises analyzing, including aligning and/or comparing the genome-wide methylation sequence reads thus obtained to genome-wide methylation sequence reads of a reference genome at the plurality of transcription factor binding sites. In some embodiments, the method comprises determining alteration in methylation status at the plurality of transcription factor binding sites in the total DNA obtained from the cell-free biological sample of the subject suffering from prostate cancer based on the analysis. In some embodiments, the method comprises generating a summary methylation profile. In some embodiments, the summary methylation profile comprises a pattern of the total DNA that is methylated and/or unmethylated at the plurality of transcription binding sites in the total DNA obtained from the cell-free biological sample of the subject. In some embodiments, the method further comprises generating a metric/quantitative score to determine the molecular phenotype of the prostate cancer.

In some embodiments, the cell-free biological sample comprises blood, plasma, and/or a bodily fluid sample. In some embodiments, the reference genome comprises a reference genome-wide methylation profile obtained from at least one biological sample obtained from a subject known to have the molecular phenotype of the prostate cancer. In an embodiment, the reference genome comprises a reference genome-wide methylation profile obtained from at least one biological sample obtained from a healthy subject. In a related embodiment, the at least one biological sample obtained from a subject known to have the molecular phenotype of the prostate cancer and/or from a healthy subject comprises a cell-free biological sample.

In a related embodiment, the step of determining genome-wide methylation profile at a plurality of transcription binding sites in the total DNA comprises treating the total DNA with a first agent that modifies methylated cytosine residues in the total DNA. In some embodiments, the method comprises subjecting the total DNA with the modified methylated cytosine to a second agent to convert unmodified cytosines to obtain uracil comprising DNA strands; amplifying the uracil comprising DNA strands; and sequencing the amplified DNA to obtain a library. In some embodiments, the method further comprises obtaining a respective normalized value for each of the plurality of transcription binding sites by aligning the library with a control genome to obtain genome-wide methylation sequence reads.

In some embodiments, the prostate cancer is a Metastatic castration-resistant prostate cancer. In some embodiments, the prostate cancer lacks androgen receptor and is characterized by gain of stem-like and neuroendocrine features.

In some embodiments, the therapeutic agent is a chemotherapeutic agent. In an embodiment, the chemotherapeutic agent is selected from a) an anti-hormone treatment; b) a cytotoxic agent; c) a biologic, preferably an antibody and/or a vaccine; and d) a targeted therapeutic agent. In an embodiment, the method further comprises administering to the subject at least one additional therapeutic modality. In some embodiments, the at least one additional therapeutic modality comprises surgery and/or radiation.

In another embodiment, the present disclosure relates to a method comprising obtaining total DNA from a cell-free biological sample. In some embodiments, the method comprises modifying the total DNA and amplifying the modified total DNA to obtain a genome-wide methylation profile at a plurality of transcription binding sites in the total DNA. In an embodiment, the method comprises analyzing, including aligning and/or comparing the genome-wide methylation profile obtained to a reference genome-wide methylation profile at the plurality of transcription binding sites in DNA obtained from a reference biological sample. In an embodiment, the method comprises analyzing, including aligning and/or comparing the genome-wide methylation profile obtained to a reference genome-wide methylation profile at the plurality of transcription binding sites in total DNA obtained from a cell-free reference biological sample. In some embodiments, the method comprises determining alteration in methylation status at the plurality of transcription binding sites based on the analysis. In an embodiment, the method comprises generating a summary methylation profile for each of the plurality of transcription binding sites, where the summary methylation profile comprises a pattern of the total DNA that are methylated and/or unmethylated at the plurality of transcription binding sites in the total DNA obtained from the cell-free biological sample. In some embodiments, the method comprises generating a quantitative/metric score. In some embodiments, the metric/quantitative score comprises generating a Transcription Factor Activity Score (TFAScore). In some embodiments, the cell-free biological sample is selected from blood, plasma, and a bodily fluid obtained from a subject suffering from prostate cancer. In some embodiments, the TFAScore is indicative of genome-wide activity and/or binding of transcription factors in the cell-free sample.

In an embodiment, the reference genome-wide methylation profile comprises a genome-wide methylation profile at the plurality of transcription binding sites in DNA obtained from at least one biological sample of a subject known to have a specific type of prostate cancer. In some embodiments, the DNA obtained from at least one biological sample of a subject known to have a specific type of prostate cancer comprises genomic DNA. In some embodiments, the at least one biological sample of a subject known to have a specific type of prostate cancer comprises a cell-free biological sample. In some embodiments, the reference genome-wide methylation profile comprises a genome-wide methylation profile at the plurality of transcription binding sites in DNA obtained from at least one biological sample of a healthy subject. In some embodiments, the DNA obtained from at least one biological sample of a healthy subject comprises genomic DNA. In some embodiments, the at least one biological sample of a healthy subject comprises a cell-free biological sample.

In some embodiments, the present disclosure relates to a method of treatment of prostate cancer. In an embodiment, the method comprises determining a type of prostate cancer by analyzing total DNA obtained from a cell-free biological sample of a subject, wherein the subject is a human. In some embodiments, the method further comprises selecting at least one therapeutic modality based on the type of prostate cancer. In some embodiments, the method comprises administering to the subject an effective amount of the at least one therapeutic modality. In an embodiment, the method comprises performing methylation-aware genome-wide sequencing for the total DNA obtained from a cell-free biological sample of the subject to generate genome-wide sequence reads, wherein the genome-wide sequence reads comprise a methylation status at a plurality of transcription binding sites of the total DNA. In some embodiments, the method comprises analyzing, including aligning and/or comparing to a reference human genome the genome-wide sequence reads obtained to identify each of a plurality of transcription binding sites, and using the analysis to obtain methylation status for each of the plurality of transcription binding sites in the total DNA. In some embodiments, the method comprises generating a summary methylation profile from the methylation status for each of the plurality of transcription binding sites. In some embodiments, the summary methylation profile comprises a pattern of the total DNA that is methylated and/or unmethylated at the plurality of transcription binding sites in the total DNA obtained from the cell-fee biological sample of the subject. In some embodiments, the method comprises determining the subject has a specific type of prostate cancer based at least in part on the summary methylation profile. In some embodiments, the step of determining the type of prostate cancer comprises analyzing differences between values of the summary methylation profile obtained for the total DNA of the subject and values of one or more reference methylation profiles. In some embodiments, at least one of the one or more reference methylation profiles is obtained from DNA of at least one biological sample obtained from a subject known to have the specific type of prostate cancer and/or a healthy subject. In some embodiments, the DNA of at least one biological sample obtained from a subject known to have the specific type of prostate cancer and/or a healthy subject comprises genomic DNA. In some embodiments, the at least one biological sample is a cell-free sample and the DNA obtained comprises total DNA.

In some embodiments, a computer-implemented method of determining a phenotype in a sample from a subject is provided. A computing system receives sequencing data for the sample. The computing system aligns the sequencing data to a reference genome to generate alignment data. The computing system processes the alignment data to create methylation data. The computing system processes the methylation data to create summary methylation data for transcription factor binding sites associated with a first phenotype of interest and a second phenotype of interest. The computing system determines a first transcription factor activity score for the first phenotype of interest and a second transcription factor activity score for the second phenotype of interest based on the summary methylation data. The computing system determines the phenotype in the sample based on the first transcription factor activity score and the second transcription factor activity score.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic overview of distinct clinically relevant molecular phenotypes of advanced prostate cancer. AR denotes androgen receptor; NE denotes neuroendocrine differentiation.

FIG. 2. shows AR Transcription Factor binding sites (TFBSs) methylation status in AR+ and AR− tumors with separation of tumor samples in distinct groups.

FIG. 3 shows pattern of transcription factor binding site methylation alterations in advanced prostate cancer models.

FIG. 4 demonstrates an estimation of cfDNA NEPC fraction in cases with pure AR+/NE− (ARPC) and AR−/NE+(NEPC) molecular phenotype.

FIG. 5 depicts prostate cancer with divergent molecular phenotypes in different metastatic sites (Left panel) and deconvolution of molecular subtypes from cfDNA using TFAScore (Right panel).

FIG. 6 is a flow chart depicting the integrated approach of the present disclosure useful for determining cancer molecular subtypes based on genome-wide analysis of methylation pattern from DNA obtained from tissue and/or cfDNA samples.

FIG. 7 is a flowchart that illustrates a non-limiting example embodiment of a method of determining a phenotype in a sample from a subject according to various aspects of the present disclosure.

FIG. 8 is a block diagram that illustrates aspects of a non-limiting example embodiment of a computing device appropriate for use with the present disclosure.

FIG. 9 shows TFAScores for AR and ASCL1 binding sites for ARPC (n=6) and NEPC (n=2) mCRPC cfDNA samples. Differential tumor fraction normalized AR and ASCL1 TFAScores−ΔTFx (AR/ASCL1).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “Transcription Factor Activity Score” (TFAScore) generally refers to a measure for the activity of each transcription factor (TF) based on the methylation pattern at its respective Transcription Binding Site (TFBS) binding site. The TFAScore may be used to objectively compare the binding activity of a plurality of transcription factors to their respective TFBSs in serial analyses from the same subject or among different individuals. This score provides a robust assessment of transcription factor binding activity with particular utility to use cfDNA in clinical diagnostics, cancer detection and treatment monitoring.

As used herein, the term “aligned sequence pattern” generally refers to a spatial pattern of sequence reads after alignment to a reference genome.

As used herein, the term “circulating free DNA” or “cell-free DNA” (cfDNA) generally refers to deoxyribonucleic acid (DNA) that was first detected in human blood plasma in 1948. (Mandel, P. Metais, P., C R Acad. Sci. Paris, 142, 241-243 (1948)). Since then, its connection to disease has been established in several areas. (Tong, Y. K. Lo, Y. M., Clin Chim Acta, 363, 187-196 (2006)). Studies reveal that much of the circulating nucleic acids in blood arise from necrotic or apoptotic cells (Giacona, M. B., et al., Pancreas, 17, 89-97 (1998)) and greatly elevated levels of nucleic acids from apoptosis is observed in diseases such as cancer. (Giacona, M. B., et al., Pancreas, 17, 89-97 (1998); Fournie, G. J., et al., Cancer Lett, 91, 221-227 (1995)). Particularly for cancer, where the circulating DNA bears hallmark signs of the disease including mutations in oncogenes, microsatellite alterations, and, for certain cancers, viral genomic sequences, DNA or RNA in plasma has become increasingly studied as a potential biomarker for disease. 16266-16271 (2008)). Thus, cfDNA comprises total DNA obtained from cell-free biological samples and/or represents genomic DNA and circulating tumor DNA (ctDNA).

As used herein, the term “diagnoses” or “diagnosis” of a status or outcome generally refers to predicting or diagnosing the status or outcome, determining predisposition to a status or outcome, monitoring treatment of a subject (e.g., a patient), diagnosing a therapeutic response of a subject (e.g., a patient), and prognosis of status or outcome, progression, and response to particular treatment.

As used herein, the term “nucleic acid” generally refers to a polynucleotide comprising two or more nucleotides. It may be DNA or RNA. The nucleic acid may be a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include deoxyribonucleic (DNA), ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent. A “variant” nucleic acid is a polynucleotide having a nucleotide sequence identical to that of its original nucleic acid except having at least one nucleotide modified, for example, deleted, inserted, or replaced, respectively. The variant may have a nucleotide sequence at least about 80%, 90%, 95%, or 99%, identity to the nucleotide sequence of the original nucleic acid.

As used herein, the terms “amplifying” and “amplification” generally refer to increasing the size or quantity of a nucleic acid molecule. The nucleic acid molecule may be single-stranded or double-stranded. Amplification may include generating one or more copies or “amplified product” of the nucleic acid molecule. Amplification may be performed, for example, by extension (e.g., primer extension) or ligation. Amplification may include performing a primer extension reaction to generate a strand complementary to a single-stranded nucleic acid molecule, and in some cases generate one or more copies of the strand and/or the single-stranded nucleic acid molecule. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase.

The term “transcription factor” generally refers to a protein that controls the rate of transcription of genetic information from DNA to messenger RNA by binding to a specific DNA sequence. Transcription factors are proteins that bind to DNA-regulatory sequences (e.g., enhancers and silencers), usually localized in the 5′-upstream region of target genes, to modulate the rate of gene transcription. This may result in increased or decreased gene transcription, protein synthesis, and subsequent altered cellular function, (for example, cells changing, in response to the environment (normal or pathological), for example during atrophy, hypertrophy, hyperplasia, metaplasia, or dysplasia). As used herein, specific transcription factors are referred to by a nomenclature although other synonyms may also be used for the transcription factors recited herein.

As used herein, the term “subject” generally refers to an individual, entity or a medium that has or is suspected of having testable or detectable genetic information or material. A subject can be a person, individual, or patient. The subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a cancer or a stage of a cancer of the subject. As an alternative, the subject can be asymptomatic with respect to such health or physiological state or condition.

As used herein a “biological sample” may be cell-free biological sample or a substantially cell-free biological sample or may be processed or fractionated to produce a cell-free biological sample. For example, cell-free biological samples may include cell-free ribonucleic acid (cfRNA), cell-free deoxyribonucleic acid (cfDNA), cell-free protein and/or cell-free polypeptides. A biological sample may be tissue (e.g., tissue obtained by biopsy), blood (e.g., whole blood), plasma, serum, sweat, urine, saliva, cerebrospinal fluid (CSF), lung lavage fluid, or a derivative thereof. Cell-free biological samples may be obtained or derived from subjects using an ethylenediaminetetraacetic acid (EDTA) collection tube, a cell-free RNA collection tube (e.g., Streck), or a cell-free DNA collection tube (e.g., Streck). Cell-free biological samples may be derived from whole blood samples by fractionation. Biological samples or derivatives thereof may contain cells. For example, a biological sample may be a blood sample or a derivative thereof (e.g., blood collected by a collection tube or blood drops), a tumor sample, a tissue sample, a urine sample, or a cell (e.g., tissue) sample.

In some embodiments, the cell-free fraction or cell-free biological sample may be blood serum or blood plasma. The term “cell-free fraction” of a biological sample, as used herein, generally refers to a fraction of the biological sample that is substantially free of cells. As used herein, the term “substantially free of cells” generally refers to a preparation from the biological sample comprising fewer than about 20,000 cells per mL, fewer than about 2,000 cells per mL, fewer than about 200 cells per mL, or fewer than about 20 cells per mL.

The present disclosure relates to methods and compositions for detecting and diagnosing cancer for the effective clinical management of the disease that involves the detection of epigenetic changes on a genome wide basis. Also, disclosed herein are methods for selecting cancer patients in accordance with the disclosed methods, as well as methods for treating cancer patients.

By “epigenetic change” is meant a modification of a genomic locus caused by an epigenetic mechanism, such as a change in methylation status, other modifications to C-5 position of the cytosine ring, chromatin accessibility, or histone modification for example. Frequently, an epigenetic change will result in an alteration in the levels of expression of the gene which may be detected (at the RNA or protein level as appropriate) as an indication of the epigenetic change. Often epigenetic change results in silencing or down regulation of the gene, referred to herein as “epigenetic silencing”. The most frequently investigated epigenetic change involves determining the methylation status of genes, where an increased level of methylation is typically associated with the relevant cancer (since it may cause down regulation of gene expression). It is now widely accepted that cellular malfunction and gene silence can result from both genomic and epigenetic alterations. These two systems work in concert to promote tumor growth and malignancy.

A “site” corresponds to a single site, which may be a single base position or a group of correlated base positions, e.g., a CpG site. A “locus” may correspond to a region that includes multiple sites. A locus can include just one site, which would make the locus equivalent to a site in that context.

The “methylation index” for each genomic site (e.g., a CpG site) refers to the proportion of signal derived from a methylated CpG. For sequencing based methods, this is the methylation index in the percent of sequence reads showing methylation at the site over the total number of reads covering that site. The “methylation density” of a region is the number of reads at sites within the region showing methylation divided by the total number of reads covering the sites in the region. The sites may have specific characteristics, e.g., being CpG sites. Thus, the “CpG methylation density” of a region is the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after, for e.g., when methylation pattern is determined by bisulfite treatment (which corresponds to methylated cytosine) at CpG sites as a proportion of all CpG sites covered by sequence reads mapped to the 100-kb region. This analysis can also be performed for any bin size. A region could be the entire genome or a chromosome or part of a chromosome (e.g., a chromosomal arm). The methylation index of a CpG site is the same as the methylation density for a region when the region only includes that CpG site. The “proportion of methylated cytosines” refers the number of cytosine sites, “C's”, that are shown to be methylated (for example unconverted after bisulfite conversion) over the total number of analyzed cytosine residues, i.e., including cytosines outside of the CpG context, in the region. The methylation index, methylation density and proportion of methylated cytosines are examples of “methylation levels” and/or methylation status”.”

A “methylation profile” and/or “summary methylation profile” (also called methylation pattern) includes information related to DNA methylation for a region or a collection of regions. Information related to DNA methylation can include, but is not limited to, a methylation index of a CpG site, a methylation density of CpG sites in a region, a distribution of CpG sites over a contiguous region, a pattern or level of methylation for each individual CpG site within a region that contains more than one CpG site, and non-CpG methylation. The latter can involve the methylation of cytosine that precede a base other than G, including A, C or T. A methylation profile of a substantial part of the genome can be considered equivalent to the methylome. “DNA methylation” in mammalian genomes typically refers to the addition of a methyl group to the 5′ carbon of cytosine residues (i.e., 5-methylcytosines) among CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported.

As used herein, the phrase “therapeutic agent” refers to any agent that has a therapeutic effect and/or elicits a desired biological and/or pharmacological effect, when administered to a subject. In some embodiments, an agent is considered to be a therapeutic agent if its administration to a relevant population is statistically correlated with a desired or beneficial therapeutic outcome in the population, whether or not a particular subject to whom the agent is administered experiences the desired or beneficial therapeutic outcome.

As used herein, the term “therapeutically effective amount” refers to an amount of an agent which confers a therapeutic effect on a treated subject, at a reasonable benefit/risk ratio applicable to any medical treatment. A therapeutic effect may be objective (i.e., measurable by some test or marker) or subjective (i.e., subject gives an indication of or feels an effect). In particular, a “therapeutically effective amount” refers to an amount of a therapeutic agent effective to treat, ameliorate, or prevent a desired disease or condition, or to exhibit a detectable therapeutic or preventative effect, such as by ameliorating symptoms associated with a disease, preventing or delaying onset of a disease, and/or also lessening severity or frequency of symptoms of a disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, on combination with other agents. Also, a specific therapeutically effective amount (and/or unit dose) for any particular patient may depend upon a variety of factors including what disorder is being treated; disorder severity; activity of specific agents employed; specific composition employed; age, body weight, general health, and diet of a patient; time of administration, route of administration; treatment duration; and like factors as is well known in the medical arts.

A “therapeutic regimen”, as that term is used herein, refers to a dosing regimen whose administration across a relevant population is correlated with a desired or beneficial therapeutic outcome.

As used herein, the term “treatment” (also “treat” or “treating”) refers to any administration of a substance that partially or completely alleviates, ameliorates, relives, inhibits, delays onset of, reduces severity of, and/or reduces frequency, incidence or severity of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

DNA hypermethylation is an epigenetic alteration in which the addition of methyl groups (CH3) to particular DNA cytosines regulates the activation of the genes. Specifically, the cytosine of the CpG dinucleotides (CpG islands), which are enriched in the promoter regions and introns of human genes. Methylation of the cytosine residues in the promoter region of a gene in the context of CpG islands correlates with silencing of the gene. Numerous malignancies, including lung, breast, ovarian, renal, cervical, prostate, and colorectal cancer, have been linked to DNA hypermethylation. DNA methylation patterns in cancer cells differ dramatically from those in healthy cells. Therefore, distinguishing cancer cells from normal cells can be accomplished by detecting methylation patterns in the genome of cancer cells. DNA methylation patterns have shown promise in cancer diagnosis providing for an accurate subclassification of cancers for clinical management. There are potentially millions of DNA methylation markers, and it is challenging to determine which ones are biologically informative for a particular disease state. The inventors by way of this disclosure demonstrate that DNA methylation patterns at sites of transcription factor binding can be used to not only determine transcription factor activity but also for the development of biologically informed cancer biomarkers. The inventors have applied this approach to advanced prostate cancer to accurately subclassify prostate cancer into clinically relevant subtypes by analyzing genome-wide methylation status at transcription binding sites in tissue and cell-free samples of prostate cancer.

DNA methylation markers offer certain advantages when compared to other biochemical markers. Specifically, an important advantage is that DNA alterations often precede apparent malignant changes and thus may be of use in early diagnosis of cancer. Since DNA is much more stable and, unlike protein, can be amplified by powerful amplification-based techniques for increased sensitivity, it offers applicability for situations where sensitive detection is necessary, such as when tumor DNA is scarce or diluted by an excess of normal DNA. Bodily fluids provide a cost-effective and early non-invasive procedure for cancer detection.

Over the past years, there has been increased interest in assessing tumor relevant alterations by analyzing tumor derived cell-free DNA (cfDNA), for example DNA obtained from blood samples, plasma, or feces. cfDNA is DNA released from cells including tumor cells into circulation and is a non-invasive solution for addressing challenges in sampling, tumor representation and cost. Emerging approaches to analyze genetic and epigenetic alterations from cfDNA have demonstrated their application for analyzing prostate cancer (PC) molecular features. These methods, however, include separate specialized assays that measure epigenetic features such as chromatin accessibility and DNA methylation. Previous workflows therefore lack multi-omic integration of these epigenetic features. However, performing separate assays is cost prohibitory and inefficient use of an already limited sample (cell-free sample). The present disclosure overcomes these limitations by providing an integrated approach useful for determining cancer molecular subtypes based on genome-wide analysis of methylation pattern from DNA obtained from tissue and/or cfDNA samples.

DNA methylation changes are nearly universal in human prostate cancer, can mediate epigenetic control of key oncogenic transcriptional programs and show exquisite biomarker properties. From an analytical perspective, DNA methylation represents extremely stable analytes and compared to genomic alterations, methylation alterations are significantly easier to detect even in highly dilute conditions. While single-gene centric analytical approaches to assess DNA methylation changes have been used in the past, only a small number of studies have applied genome wide analyses to cfDNA in prostate cancer (PC) patients. Since genome-wide analyses allow for the extraction of methylation data from a large number of genomic loci, they are inherently more sensitive and less prone to inter-sample variability. In addition, from genome wide analyses a larger number of features can be extracted making these approaches more versatile and broadly applicable. Lastly, with the decreasing costs of genome wide analyses workflows, they represent a cost-effective alternative to targeted assays.

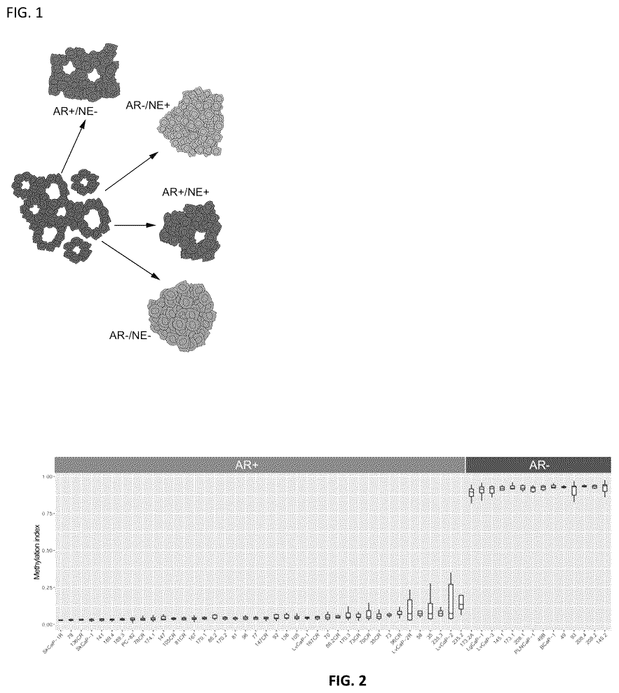

Metastatic castration-resistant prostate cancer (mCRPC) is a heterogeneous disease which can be classified into clinically relevant subtypes based on the expression of transcription factors (TF), such as the androgen receptor (AR) and neuroendocrine markers (FIG. 1). Neuroendocrine prostate cancer (NEPC), characterized by gain of stem-like and neuroendocrine features and lack of AR expression is a clinically aggressive variant. Due to the absence of adequate biomarkers, NEPC is usually detected at a very advanced stage. There is mounting evidence that molecular subtype changes seen in NEPC are enforced by widespread epigenetic alterations, in particular DNA methylation changes.

The inventors have developed a novel quantitative approach, the TFAScore (Transcription Factor Activity score) an integrated tool/metric to determine prostate cancer molecular subtypes from tissue and cfDNA samples based on comparison of genome-wide methylation patterns of transcription factor binding sites (TFBS) in total DNA obtained from cancer and healthy subjects. The TFAScore disclosed herein is a metric that may be used to objectively compare the methylation pattern of TFBSs in serial analyses of biological samples obtained from the same subject or among different subjects. The TFAScore determines the proportion of tissue where the respective Transcription Factor (TF) is active. This involves analyzing the methylation pattern around a plurality of TFBSs ranging from several hundreds to thousands in the total DNA obtained from the biological sample. These TFBSs can be further selected for a given tissue type to increase specificity of TFAScore.

While numerous previously described DNA methylation-based assays have focused on the early detection of prostate cancer or the assessment of tumor burden using liquid biopsies, TFAScore differs in several important aspects from the prior work which focused on using DNA methylation alterations solely for tumor detection by measuring transcription factor markers. In particular, the present disclosure provides an integrated analysis workflow that can detect clinically relevant molecular phenotypes of advanced metastatic prostate cancers by identifying and utilizing phenotype-specific transcription factor markers. Clinical phenotypes relate to the activity of key driver transcription factors that define tumors with distinct responses to standard of care therapies. For example, tumors with activity of the androgen receptor (AR) show responses to AR-directed therapies. This includes drugs inhibiting the gonadal or extragonadal production of androgens (e.g., leuprolide, goserelin, triptorelin, histrelin and abiraterone acetate) as well as AR antagonists (e.g., biculatamide, enzalutamide, darolutamide and apalutamide). Tumors which show no AR activity are unresponsive to AR-blockade. Therefore, biomarkers that determine AR activity (such as TFAScore disclosed herein) can predict responses to AR-directed therapies. Similarly, tumors with neuroendocrine differentiation, which show activity of the master neuronal transcription factor ASCL1 are known to respond to platinum-based chemotherapy (e.g., cisplatin, carboplatin, oxaliplatin). Thus, the accurate assessment of transcription factor activity genome-wide can be used to guide treatment decisions. The molecular phenotypes detected by the methods of the present disclosure are not simply diagnostic (detecting absence of presence of cancer) or prognostic (associated with outcomes) but are predictive of responses to therapy. Advantageously, the methods disclosed herein allow for the generation of clinically actionable data reflective of underlying disease biology.

The present disclosure relates to a method of generating a TFAScore from tissue and cfDNA samples using genomic and circulating DNA/total DNA, respectively, obtained from biological samples of a subject. The methodology utilized to generate TFAScore relies on genome-wide methylation mapping to detect genome-wide transcription factor binding profiles in biological samples. Gene expression patterns are controlled by the actions of transcription factors (TFs) at their DNA binding sites. The most popular method for Transcription factor (TF) profiling is chromatin immunoprecipitation (ChIP), which has undergone little alteration since it was first published more than 30 years ago. ChIP involves formaldehyde crosslinking of cells, chromatin fragmentation and solubilization, antibody addition, and recovery of the antibody-bound chromatin for DNA extraction. The usage of X-ChIP (formaldehyde crosslinking ChIP) has been transformed by subsequent developments in DNA mapping methods, and single base-pair resolution mapping of TFs is now possible using ChIP-seq.

The inventors analyzed genome-wide methylation patterns in 60 prostate cancer patient-derived xenograft (PDX) and 133 mCRPC tumors using array- and sequencing-based assays and integrated DNA methylation with transcription factor (TF) cistrome data from public repositories (for example, cistrome.org/db and/or gtrd.biouml.org) and previously published studies to determine the landscape of methylation alterations at key lineage TF binding sites (TFBS), including androgen receptor (AR) and Achaete-Scute Family BHLH Transcription Factor 1 (ASCL1), whereby the methylation level/status at each annotated Transcription factor binding site (TFBS) was determined across the genome. To perform this analysis methylation at each transcription factor binding site (TFBS) was obtained using a custom tool.

Briefly, the custom tool uses the raw methylation data (for e.g., bisulfite converted sequencing data) obtained from each biological sample analyzed as input in FASTA format. A program (for e.g., Bismarck) is used to align/compare the biological sample DNA methylation data to a reference genome (for e.g., BAM format) to perform methylation calls in a single step. The data thus obtained is then used to generate methylation value files (methylation value at every genomic locus) in BedGraph and BigWig format. For array-based sequence data similar methylation value files are generated using appropriate tools designed for respective arrays. For example, for Human MethylationEPIC microarray platform minfi package is used to generate methylation value files. Next, the methylation value files are provided as input to a custom tool along with TFBSs for each TF in bed file format. The custom tool extracts methylation values for a given window (usually 2 kbp) for each TFBS for each TF for every sample. It then generates summary methylation profile for each TF by calculating summary metrics such as mean, inter quartile ranges, standard deviation among others. This summary methylation profile is generated from pure molecular phenotypes tissue or plasma cfDNA, normal (healthy) plasma cfDNA and immune and other tissue (such as normal liver, bone etc.). These summary methylation profiles are then used to develop a mixture model where the model estimates the proportion of molecular phenotype using the marker TF in a given biological sample (tissue or cfDNA). This estimate is referred to as the TFAScore.

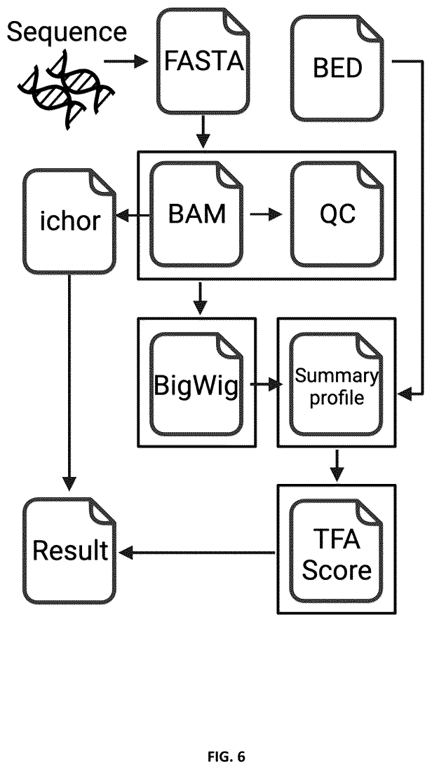

This model was then used to discern tumor molecular phenotypes from cfDNA in three independent cohorts of mCRPC patients using low-pass whole genome bisulfite sequencing and enzymatic methyl-sequencing (EM-seq) method. The inventors demonstrate herein that methylation patterns at TFBSs can determine TF activity and can be used to classify molecular subtypes from both tumor tissue and cfDNA. The inferred TF activity based on the TFAScore was corroborated by gold-standard protein assays (western blot, immunohistochemistry). Specifically, for prostate cancer, this approach can accurately detect NEPC by cost-effective low-pass EM-seq. More broadly, the present disclosure provides a novel analysis framework for robustly assessing molecular tumor phenotypes in cfDNA with applications in solid and liquid tumor diagnostics. FIG. 6 shows a flow chart depicting the integrated approach of the present disclosure useful for determining cancer molecular subtypes based on genome-wide analysis of methylation pattern from DNA obtained from tissue and/or cfDNA samples.

Thus, in some aspects the present disclosure pertains to a novel DNA methylation-based method for molecular subtyping and disease monitoring from biological samples obtained from a subject. In some embodiments, the biological samples comprise cell-free biological samples. In some embodiment, the methods disclosed herein comprise obtaining DNA from the cell-free biological samples. In some embodiments, the DNA obtained from the cell-free biological samples comprise cell-free DNA (cfDNA) or total DNA. In an embodiment, the cfDNA or total DNA comprises genomic DNA and circulating tumor DNA (ctDNA). In some embodiments, the methods disclosed herein can be used for assessing methylation pattern in DNA obtained from tissue. The methods disclosed herein can be employed to analyze cfDNA or total DNA and/or tissue DNA from virtually any organism, including, but not limited to, plants, animals (e.g., reptiles, mammals, insects, worms, fish, etc.), tissue samples, bacteria, fungi (e.g., yeast), phage, viruses, cadaveric tissue, archaeological/ancient samples, etc. In certain embodiments, the total DNA used in the method may be derived from a mammal, where in certain embodiments the mammal is a human. In exemplary embodiments, the biologic sample may contain DNA from a mammalian cell, such as, a human, mouse, rat, or monkey cell. The biologic sample may be made from blood or blood products, cultured cells, formalin fixed samples or cells of a clinical sample, e.g., a tissue biopsy (for example from a cancer), scrape or lavage or cells of a forensic sample (i.e., cells of a sample collected at a crime scene). In some embodiments, the total DNA may be obtained from a biological sample such as bodily fluids and stool. Bodily fluids of interest include but are not limited to, blood, serum, plasma, saliva, mucous, phlegm, cerebral spinal fluid, pleural fluid, tears, lacteal duct fluid, lymph, sputum, cerebrospinal fluid, synovial fluid, urine, amniotic fluid, and semen. In some embodiments, a sample may be obtained from a subject, e.g., a human. In some embodiments, the biologic sample analyzed may be a sample of cell-free DNA obtained from blood (serum or plasma), or urine.

In a further aspect, the present disclosure relates to a method of treatment of prostate cancer. In some embodiments, the method comprises determining a molecular phenotype of prostate cancer. In some embodiment, the method further comprises administering to the subject an effective amount of a therapeutic agent based on the determination of the molecular phenotype of the prostate cancer. In some embodiments, the method of determining the molecular phenotype of the prostate cancer comprises obtaining a cell-free biological sample from a subject suffering from prostate cancer. In an embodiment, the method comprises isolating/obtaining total DNA from the cell-free biological sample of the subject. In some embodiments, the method comprises determining genome-wide methylation profile at a plurality of transcription factor binding sites in the total DNA of the subject suffering from prostate cancer to obtain genome-wide methylation sequence reads. In an embodiment, the method comprises comparing the genome-wide methylation sequence reads obtained to genome-wide methylation sequence reads of a reference methylation profile comprising genome-wide methylation profile at the plurality of transcription factor binding sites. In some embodiments, the method comprises determining alteration in methylation status at the plurality of transcription factor binding sites in the total DNA of the subject suffering from prostate cancer based on the comparison; and generating a summary methylation profile. In some embodiments, the summary methylation profile comprises a pattern of the total DNA that are methylated and/or unmethylated at the plurality of transcription binding sites in the total DNA of the cell-free biological sample. In some embodiments, the method further comprises generating a quantitative value or a metric to determine the molecular phenotype of the prostate cancer. In some embodiments, generating the metric/quantitative score comprises generating a Transcription Factor Activity Score (TFAScore). In an embodiment, the TFAScore is indicative of genome-wide activity and/or binding of transcription factors in the cell-free biological sample.

In some embodiments, the therapeutic agent comprises a chemotherapeutic agent for treating prostate cancer in a subject, wherein the subject is selected for treatment based on methods described and disclosed herein. In certain embodiments the chemotherapeutic agent comprises, consists essentially of or consists of: a) an anti-hormone treatment; b) a cytotoxic agent; c) a biologic, preferably an antibody and/or a vaccine; and/or d) a targeted therapeutic agent. In some embodiments, the treatment with a chemotherapeutic agent may be combined with other therapeutic agents and/or therapeutic modalities. In some embodiments, the other therapeutic agents and/or therapeutic modalities may comprise radiothereapy and/or surgical treatment.

An anti-hormone treatment (or hormone therapy) is a form of treatment which reduces the level and/or activity of selected hormones, in particular testosterone. The hormones may promote tumor growth and/or metastasis. The anti-hormone treatment may comprise a luteinizing hormone blocker, such as goserelin (also called Zoladex), buserelin, leuprorelin (also called Prostap), histrelin (Vantas) and triptorelin (also called Decapeptyl). The anti-hormone treatment may comprise a gonadotrophin release hormone (GnRH) blocker such as degarelix (Firmagon) or an anti-androgen such as flutamide (also called Drogenil) and bicalutamide (also called Casodex). An exemplary anti-hormone treatment may include but is not limited to bicalutamide and/or abiraterone.

A cytotoxic agent may be a platinum-based agent and/or a taxane. Exemplary platinum-based agents include but are not limited to cisplatin, carboplatin and oxaliplatin. Exemplary taxanes include but are not limited to Docetaxel, cabazitaxel, or paclitaxel. Other cytotoxic agents contemplated by the present disclosure include vinca alkaloids like vinorelbine or vinblastine, topoisomerase inhibitors like etoposide, anthracyclines (antibiotic) like doxorubicin, or alkylating agents like estramustine.

A biologic comprises a medicinal product that is created by a biological process. A biologic may be, for example, a vaccine, blood or blood component, cells, gene therapy, tissue, or a recombinant therapeutic protein. Optionally the biologic is an antibody and/or a vaccine.

A targeted therapeutic agent comprises a therapeutic agent directed towards a specific drug target for the treatment of prostate cancer. In specific embodiments this may mean inhibitors directed towards targets such as PARP, AKT, MET, VEGFR etc. PARP inhibitors are a group of pharmacological inhibitors of the enzyme poly ADP ribose polymerase (PARP). Several forms of cancer are more dependent on PARP than regular cells, making PARP an attractive target for cancer therapy. Exemplary PARP inhibitors include iniparib, olaparib, rucaparib, veliparib, CEP 9722, MK 4827, BMN-673 and 3-aminobenzamide. AKT, also known as Protein Kinase B (PKB), is a serine/threonine-specific protein kinase that plays a key role in multiple cellular processes such as glucose metabolism, apoptosis, cell proliferation, transcription and cell migration. AKT is associated with tumor cell survival, proliferation, and invasiveness. Exemplary AKT inhibitors include VQD-002, Perifosine, Miltefosine and AZD5363. MET is a proto-oncogene that encodes hepatocyte growth factor receptor (HGFR). The hepatocyte growth factor receptor protein possesses tyrosine-kinase activity. Exemplary kinase inhibitors for inhibition of MET include K252a, SU11274, PHA-66752, ARQ197, Foretinib, SGX523 and MP470. MET activity can also be blocked by inhibiting the interaction with HGF. Many suitable antagonists including truncated HGF, anti-HGF antibodies and uncleavable HGF are known. VEGF receptors are receptors for vascular endothelial growth factor (VEGF). Various inhibitors are known such as lenvatinib, motesanib, pazopanib and regorafenib.

In certain embodiments the radiotherapy/radiation therapy is extended radiotherapy, preferably extended-field radiotherapy. Radiation therapy may use external radiation (using a machine outside the body) or internal radiation. Internal radiation involves putting radioisotopes (materials that produce radiation) through thin plastic tubes into the area where cancer cells are found. Prostate cancer is treated with external and internal (implant) radiation. Radiation therapy may be used alone or in addition to surgery.

Surgical treatment may include but is not limited to radical prostatectomy. A radical prostatectomy comprises removal of the entire prostate gland, the seminal vesicles and the vas deferens. In further embodiments surgical treatment may comprise tumor resection i.e., removal of all or part of the tumor.

In some embodiments, a therapeutic agent is administered in a therapeutically effective amount and/or according to a dosing regimen that is correlated with a particular desired outcome (e.g., with treating or reducing risk for CRPC and/or doubly resistant prostate cancer).

Doses or amounts to be administered in accordance with the present disclosure may vary, for example, depending on the nature and/or extent of the desired outcome, on particulars of route and/or timing of administration, and/or on one or more characteristics (e.g., weight, age, personal history, genetic characteristic, lifestyle parameter, or combinations thereof). Such doses or amounts can be determined by those of ordinary skill. In some embodiments, an appropriate dose or amount is determined in accordance with standard clinical techniques. Alternatively, or additionally, in some embodiments, an appropriate dose or amount is determined through use of one or more in vitro or in vivo assays to help identify desirable or optimal dosage ranges or amounts to be administered.

In some examples, summary methylation profiles obtained for each TF are used as input features in machine learning models to find correlations between the summary methylation profile and subject (e.g., patient) groups. Examples of such patient groups include presence of diseases or conditions, stages, subtypes, responders vs. non-responders, and progressors vs. non-progressors. In some examples, feature matrices are generated to compare samples obtained from subjects with known conditions or characteristics. In some examples, samples are obtained from healthy subjects or subjects who do not have prostate cancer, and samples from patients known to have prostate cancer.

In some exemplary embodiments, the methods disclosed herein may be used to identify the effect of a therapeutic agent, e.g., a chemotherapeutic agent, or to determine if there are differences in the effect of two or more different therapeutic agents. In these embodiments, total DNA from two or more identical populations of cells may be obtained from a subject, prepared and, depending on how the experiment is to be performed, the one or more of the populations of cells obtained from the subject may be incubated with at least one therapeutic agent for a defined period of time. In some embodiments, the subject is suffering from prostate cancer. After incubation with the at least one therapeutic agent, the total DNA from one or both of the populations of cells can be analyzed using the methods set forth herein, and the results can be compared. In a particular embodiment, the cells may be blood cells, and the cells can be incubated with the therapeutic agent ex vivo. These methods can be used to determine the mode of action and/or effectiveness of a therapeutic agent, to identify changes in chromatin structure, or transcription factor occupancy in response to the therapeutic agent, for example. In some embodiments, the method may be useful in selecting a therapeutic agent to treat a subject. In some embodiments the subject has prostate cancer. In some embodiments, the prostate cancer comprises Metastatic castration-resistant prostate cancer (mCRPC).

The method set forth herein may also be used to provide a reliable diagnostic for a disease or condition. The method can be applied to the characterization, classification, differentiation, grading, staging, diagnosis, or prognosis of a condition characterized by an epigenetic pattern. For example, the method can be used to determine whether the methylation pattern in a biological sample obtained from an individual suspected of being affected by a disease or condition is the same or different compared to a biological sample that is considered “normal” with respect to the disease or condition. In particular embodiments, the methods disclosed herein can be directed to diagnosing an individual or a subject with a condition that is characterized by an epigenetic pattern at a particular locus in a test sample, where the pattern is correlated with the condition. The methods can also be used for predicting the susceptibility of an individual or a subject to a condition.

Characterization refers to the categorization and/or assessment of prostate cancer. The term “prognosis” relates to estimating the subject's chances of surviving prostate cancer. The term “diagnosis” means determining the presence of prostate cancer.

The prognosis for prostate cancer may include, essentially include, or consist of predicting a higher chance of recurrence, according to all elements of the disclosure. The prostate cancer prognosis and/or characteristics may include, mostly include, or include forecasting a shorter time to recurrence. Clinical or biochemical recurrence are both examples of recurrence. The term “biochemical recurrence” refers to an increase in PSA levels in an individual following prostate cancer treatment. The presence of biochemical recurrence could mean that prostate cancer has not been treated effectively or has recurred.

In some embodiments, the method can provide a prognosis, e.g., to determine if a patient is at risk for recurrence and/or disease progression. Cancer recurrence is a concern relating to a variety of types of cancer. The prognostic method can be used to identify surgically treated patients likely to experience cancer recurrence so that they can be offered additional therapeutic options, including preoperative or postoperative adjuncts such as chemotherapy, radiation, biological modifiers and other suitable therapies. The ability to determine which cases of prostate cancer will respond to treatment, and to which type of treatment, would be useful in appropriate allocation of treatment resources. The various therapeutic options, as discussed above, have significantly different risks and potential side effects. An accurate prognosis would also minimize application of treatment regimens which have low likelihood of success. Such also could avoid delay of the application of alternative treatments which may have higher likelihoods of success for a particular presented case. Thus, the ability to evaluate individual prostate cases for markers which subset into responsive and non-responsive groups for treatments is very useful. The prostate cancer prognosis and/or characterization may include, essentially include, or include predicting an increased likelihood of metastasis. The spread of cancer from one organ or part to another that is not nearby is known as metastasis, often known as metastatic illness. Metastases are the new cases of illness that are so produced. Finding out whether the prostate cancer has a poor prognosis may also be a part of characterizing the disease and/or predicting its prognosis. Reduced chances of cause-specific, or cancer-specific, long-term survival are examples of a bad prognosis. A measure of net survival called cause- or cancer-specific survival depicts cancer survival in the absence of other causes of death. Cancer survivorship may be 6, 7, or 8, 9, 10, 11, 12 months or 1, 2, 3, 4, 5 etc. years. Long-term survival may be survival for 1 year, 5 years, 10 years or 20 years following diagnosis. A prostate cancer with a poor prognosis may be aggressive, fast growing, and/or show resistance to treatment.

The methods disclosed herein can also be used to determine a proper course of treatment for a patient, e.g., a patient that has prostate cancer. A course of treatment refers to the therapeutic measures taken for a patient after diagnosis or after treatment. For example, a determination of the likelihood for recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with hormonal therapy, chemotherapy, radiation, immunotherapy, biological modifier therapy, gene therapy, vaccines, and the like, or adjust the span of time during which the patient is treated.

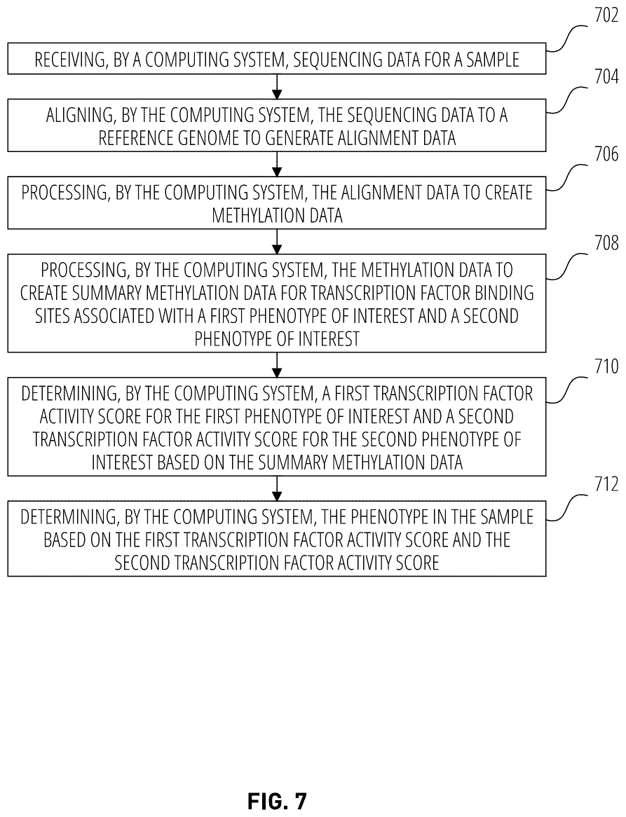

For patients with metastatic prostate cancer, the main stay of therapy includes drugs that block the gonadal or extragonadal production of androgens (e.g., leuprolide, goserelin, triptorelin, histrelin and abiraterone acetate) as well as androgen receptor (AR) antagonists (e.g., biculatamide, enzalutamide, darolutamide and apalutamide). Notably, tumors which show no AR expression/activity (around 20-30% of all advanced metastatic prostate cancer patients) are unresponsive to AR-blockade. Alternative established effective therapies for patients with AR− tumors that show neuroendocrine differentiation include platinum-based chemotherapy (e.g., cisplatin, carboplatin, oxaliplatin), which is less effective in AR+ patients. FIG. 7 is a flowchart that illustrates a non-limiting example embodiment of a method of determining a phenotype in a sample from a subject according to various aspects of the present disclosure. The method may be executed by any computing system comprising one or more computing devices and may be implemented using computer-executable instructions stored on a computer-readable storage medium that, in response to execution by one or more processors of the computing system, cause the computing system to perform the actions of the method.

At block 702, a computing system receives sequencing data for a sample (see further details described in Example 3). At block 704, the computing system aligns the sequencing data to a reference genome to generate alignment data (see further details described in Example 4). At block 706, the computing system processes the alignment data to create methylation data (see further details described in Example 4). At block 708, the computing system processes the methylation data to create summary methylation data for transcription factor binding sites (TFBSes) associated with a first phenotype of interest and a second phenotype of interest (see further details described in Example 5). At block 710, the computing system determines a first transcription factor activity score (TFAScore) for the first phenotype of interest and a second transcription factor activity score for the second phenotype of interest based on the summary methylation data (see further details described in Example 6). At block 712, the computing system determines the phenotype in the sample based on the first transcription factor activity score and the second transcription factor activity score (see further details described in Example 7).

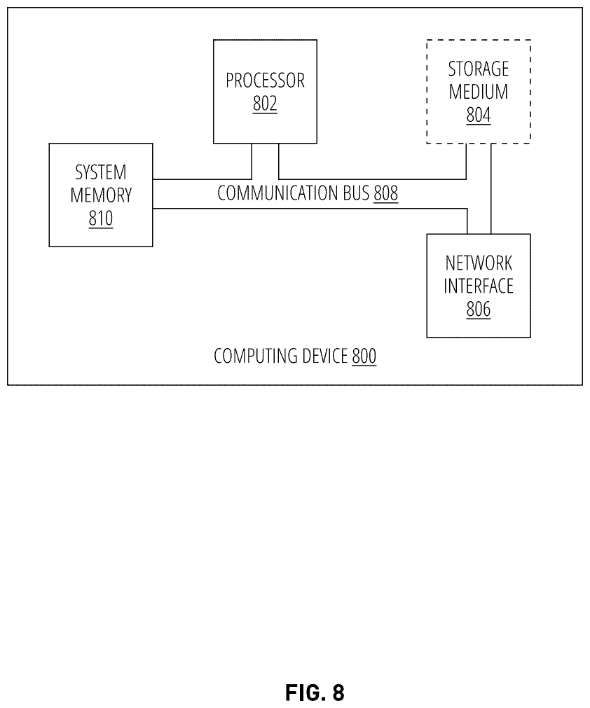

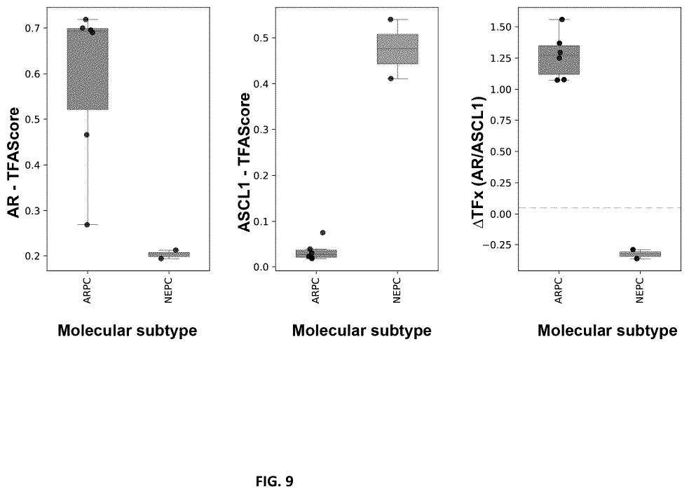

FIG. 8 is a block diagram that illustrates aspects of a non-limiting example embodiment of a computing device 800 appropriate for use with the present disclosure, such as one or more of the computing devices of a computing system used to execute the method of FIG. 7. The computing device 800 describes various elements that are common to many different types of computing devices, including but not limited to desktop computing devices, laptop computing devices, server computing devices, mobile computing devices, and cloud computing devices. While FIG. 8 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized devices. Moreover, those of ordinary skill in the art and others will recognize that the computing device 800 may be any one of any number of currently available or yet to be developed devices.

In its most basic configuration, the computing device 800 includes at least one processor 802 and a system memory 810 connected by a communication bus 808. Depending on the exact configuration and type of device, the system memory 810 may be volatile or nonvolatile memory, such as read only memory (“ROM”), random access memory (“RAM”), EEPROM, flash memory, or similar memory technology. Those of ordinary skill in the art and others will recognize that system memory 810 typically stores data and/or program modules that are immediately accessible to and/or currently being operated on by the processor 802. In this regard, the processor 802 may serve as a computational center of the computing device 800 by supporting the execution of instructions.

As further illustrated in FIG. 8, the computing device 800 may include a network interface 806 comprising one or more components for communicating with other devices over a network. Embodiments of the present disclosure may access basic services that utilize the network interface 806 to perform communications using common network protocols. The network interface 806 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 806 illustrated in FIG. 8 may represent one or more wireless interfaces or physical communication interfaces described and illustrated above with respect to particular components of the computing device 800.

In the embodiment depicted in FIG. 8, the computing device 800 also includes a storage medium 804. However, services may be accessed using a computing device that does not include means for persisting data to a local storage medium. Therefore, the storage medium 804 depicted in FIG. 8 is represented with a dashed line to indicate that the storage medium 804 is optional. In any event, the storage medium 804 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 802, system memory 810, communication bus 808, storage medium 804, and network interface 806 are known and commercially available. For ease of illustration and because it is not important for an understanding of the claimed subject matter, FIG. 8 does not show some of the typical components of many computing devices. In this regard, the computing device 800 may include input devices, such as a keyboard, keypad, mouse, microphone, touch input device, touch screen, tablet, and/or the like. Such input devices may be coupled to the computing device 800 by wired or wireless connections including RF, infrared, serial, parallel, Bluetooth, Bluetooth low energy, USB, or other suitable connections protocols using wireless or physical connections. Similarly, the computing device 800 may also include output devices such as a display, speakers, printer, etc. Since these devices are well known in the art, they are not illustrated or described further herein.

Exemplary preferred embodiments of the present disclosure include but are not limited to the following:

Embodiment 1. A method of treatment of prostate cancer comprising: (i) determining a molecular phenotype of the prostate cancer, the method comprising: (a) obtaining a cell-free biological sample from a subject suffering from prostate cancer; (b) isolating/obtaining total DNA from the cell-free biological sample of the subject; (c) determining genome-wide methylation profile at a plurality of transcription factor binding sites in the total DNA of the subject suffering from prostate cancer to obtain genome-wide methylation sequence reads at each of the plurality of transcription factor binding sites; (d) analyzing, including aligning and/or comparing the genome-wide methylation sequence reads obtained in step (c) to genome-wide methylation sequence reads of a reference genome at the plurality of transcription factor binding sites; (e) determining alterations in methylation status at the plurality of transcription factor binding sites in the total DNA of the subject suffering from prostate cancer based on the analysis in step (d); and (f) generating a summary methylation profile, wherein the summary methylation profile comprises a pattern of the total DNA that are methylated and/or unmethylated at the plurality of transcription binding sites in the total DNA of the cell-free biological sample; and (g) generating a metric/quantitative score to determine the molecular phenotype of the prostate cancer; and (ii) administering to the subject an effective amount of at least one therapeutic agent based on the determination of the molecular phenotype of the prostate cancer.

Embodiment 2. The method of claim 1, wherein the cell-free biological sample comprises blood, plasma, and/or a bodily fluid sample.

Embodiment 3. The method of embodiment 1, wherein the step of determining genome-wide methylation profile at a plurality of transcription binding sites in the total DNA comprises: (a) treating the total DNA with a first agent that modifies methylated cytosine residues in the total DNA; (b) subjecting the total DNA with the modified methylated cytosine to a second agent to convert unmodified cytosines to obtain uracil comprising DNA strands; (c) amplifying the uracil comprising DNA strands; and (d) sequencing the amplified DNA to obtain a library.

Embodiment 4. The method of embodiment 3, wherein the method further comprises obtaining a respective normalized value for the methylation status of each of the plurality of transcription binding sites in the total DNA based on a tumor fraction in the cell-free biological sample of the subject.

Embodiment 5. The method of embodiment 3, wherein the methylated cytosine residues comprise 5 methyl cytosine and 5-hydroxymethyl cytosine.

Embodiment 6. The method of embodiment 5, wherein the first agent oxidizes the methylated cytosine residues to obtain the modified methylated cytosine residues.

Embodiment 7. The method of embodiment 3, wherein the second agent is a deamination agent.

Embodiment 8. The method of embodiment 1, wherein the reference genome comprises a reference genome-wide methylation profile obtained from at least one sample obtained from a subject known to have the molecular phenotype of the prostate cancer.

Embodiment 9. The method of embodiment 1, wherein the reference genome comprises a reference genome-wide methylation profile obtained from at least one sample obtained from a healthy subject.

Embodiment 10. The method of embodiment 1, wherein the prostate cancer is a Metastatic castration-resistant prostate cancer.

Embodiment 11. The method of embodiment 10, wherein the prostate cancer lacks androgen receptor and is characterized by gain of stem-like and neuroendocrine features.

Embodiment 12. The method of embodiment 1, wherein generating the metric/quantitative score comprises generating a Transcription Factor Activity Score (TFAScore), and wherein the TFAScore is indicative of genome-wide activity and/or binding of transcription factors in the cell-free biological sample.

Embodiment 13. The method of embodiment 1, wherein the therapeutic agent is a chemotherapeutic agent.

Embodiment 14. The method of embodiment 13, wherein the chemotherapeutic agent is selected from a) an anti-hormone treatment; b) a cytotoxic agent; c) a biologic, preferably an antibody and/or a vaccine; and d) a targeted therapeutic agent.

Embodiment 15. A method comprising: (a) obtaining total DNA from a cell-free biological sample; (b) modifying the total DNA and amplifying the modified total DNA to obtain a genome-wide methylation profile at a plurality of transcription binding sites in the total DNA; (c) analyzing, including aligning and/or comparing the genome-wide methylation profile obtained in step (b) to a reference genome-wide methylation profile at the plurality of transcription binding sites in total DNA obtained from a cell-free reference sample; (d) determining alteration in methylation status at the plurality of transcription binding sites based on the analysis in step (c); (e) generating a summary methylation profile for each of the plurality of transcription binding sites, wherein the summary methylation profile comprises a pattern of the total DNA that are methylated and/or unmethylated at the plurality of transcription binding sites in the total DNA of the cell-free biological sample; and (f) generating a metric/quantitative score, wherein the metric/quantitative score comprises generating a Transcription Factor Activity Score (TFAScore).

Embodiment 16. The method of embodiment 15, wherein the cell-free biological sample is selected from blood, plasma, and a bodily fluid obtained from a subject suffering from prostate cancer.

Embodiment 17. The method of embodiment 15, wherein the reference genome-wide methylation profile comprises genome-wide methylation profile at the plurality of transcription binding sites in DNA obtained from at least one biological sample of a subject known to have a specific type of prostate cancer.

Embodiment 18. The method of embodiment 15, wherein the reference genome-wide methylation profile comprises genome-wide methylation profile at the plurality of transcription binding sites in DNA obtained from at least one biological sample of a healthy subject.

Embodiment 19. The method of embodiment 17, wherein the prostate cancer is a Metastatic castration-resistant prostate cancer.

Embodiment 20. The method of embodiment 17, wherein the prostate cancer lacks androgen receptor and is characterized by gain of stem-like and neuroendocrine features.

Embodiment 21. The method of embodiment 15, wherein the TFAScore is indicative of genome-wide activity and/or binding of transcription factors in the cell-free biological sample.

Embodiment 22. A method of treatment of prostate cancer, the method comprising: (i) determining a type of prostate cancer by analyzing total DNA obtained from a cell-free biological sample of a subject, wherein the subject is a human, the method comprising: (a) performing methylation-aware genome-wide sequencing for the total DNA to generate genome-wide sequence reads, wherein the genome-wide sequence reads comprise methylation status at a plurality of transcription binding sites of the total DNA; (b) analyzing, including aligning and/or comparing to a reference human genome the genome-wide sequence reads obtained in step (a) to identify each of a plurality of transcription binding sites, and using the analysis to obtain methylation status for each of the plurality of transcription binding sites in the total DNA; (c) generating a summary methylation profile from the methylation status for each of the plurality of transcription binding sites obtained in step (b), wherein the summary methylation profile comprises a pattern of the total DNA that are methylated and/or unmethylated at the plurality of transcription binding sites in the total DNA obtained from the cell-fee biological sample of the subject; and (d) determining the subject has a specific type of prostate cancer based at least in part on the summary methylation profile, wherein the step of determining the type of prostate cancer comprises analyzing differences between values of the summary methylation profile obtained in step (c) and values of one or more reference methylation profiles, and wherein at least one of the one or more reference methylation profiles is obtained from DNA of at least one biological sample obtained from a subject known to have the specific type of prostate cancer and/or a healthy subject; (ii) selecting at least one therapeutic modality based on the type of prostate cancer; and (iii) administering to the subject an effective amount of the at least one therapeutic modality.

Embodiment 23. A computer-implemented method of determining a phenotype in a sample from a subject, the method comprising: receiving, by a computing system, sequencing data for the sample; aligning, by the computing system, the sequencing data to a reference genome to generate alignment data; processing, by the computing system, the alignment data to create methylation data; processing, by the computing system, the methylation data to create summary methylation data for transcription factor binding sites associated with a first phenotype of interest and a second phenotype of interest; determining, by the computing system, a first transcription factor activity score for the first phenotype of interest and a second transcription factor activity score for the second phenotype of interest based on the summary methylation data; and determining, by the computing system, the phenotype in the sample based on the first transcription factor activity score and the second transcription factor activity score.

Embodiment 24. The computer-implemented method of embodiment 23, wherein determining the first transcription factor activity score includes determining a mixture weight that represents a proportion of the first phenotype of interest and a proportion of a sample background in the sample; wherein the mixture weight is used as the first transcription factor activity score.

Embodiment 25. The computer-implemented method of embodiment 24, wherein determining the mixture weight includes determining a value for a in:

P(x₁,x₂|θ)=α*P(x₁|μ₁,σ₁)+(1−α)*P(x₂|μ₂,σ₁)
wherein α is the mixture weight; wherein P(x₁|μ₁, σ₁) is a probability density function for the first phenotype of interest given a mean of the first phenotype of interest and a standard deviation of the first phenotype of interest determined from a known sample; wherein P(x₂|μ₂, σ₁) is a probability density function for the sample background given a mean of the sample background and the standard deviation of the first phenotype of interest; and wherein P(x₁, x₂|θ) is a joint probability density function for the first phenotype of interest and the sample background given features based on the summary methylation data.

Embodiment 26. The computer-implemented method of embodiment 25, wherein the features based on the summary methylation data include a mean and a standard deviation.

Embodiment 27. The computer-implemented method of any one of embodiment 24-26, wherein determining the mixture weight includes estimating the mixture weight using an Expectation-Maximization process.

Embodiment 28. The computer-implemented method of any one of embodiment 23-27, wherein processing the methylation data to create summary methylation data for transcription factor binding sites associated with the first phenotype of interest and the second phenotype of interest includes: extracting methylation values for windows around the transcription factor binding sites associated with the first phenotype of interest and the second phenotype of interest; and calculating summary metrics for the extracted methylation values.

Embodiment 29. The computer-implemented method of embodiment 28, wherein the summary metrics include one or more of a mean, an interquartile range, or a standard deviation.

Embodiment 30. The computer-implemented method of any one of embodiment 28-29, wherein the summary metrics are calculated at a plurality of bins within each window.

Embodiment 31. The computer-implemented method of embodiment 30, wherein a size of each bin of the plurality of bins is 15 bp.

Embodiment 32. The computer-implemented method of any one of embodiment 23-31, wherein determining the phenotype in the sample based on the first transcription factor activity score and the second transcription factor activity score includes: comparing the first transcription factor activity score to the second transcription factor activity score; and identifying the first phenotype of interest or the second phenotype of interest as the phenotype in the sample based on the comparison.

Embodiment 33. The computer-implemented method of embodiment 32, wherein the sample is from a tissue biopsy, and wherein comparing the first transcription factor activity score to the second transcription factor activity score includes: determining a difference between the first transcription factor activity score and the second transcription factor activity score; and comparing an absolute value of the difference to a threshold difference value; and wherein identifying the first phenotype of interest or the second phenotype of interest as the phenotype in the sample based on the comparison includes: in response to determining that the absolute value of the difference is greater than the threshold difference value, identifying the first phenotype of interest or the second phenotype of interest as the phenotype in the sample based on which transcription factor activity score is higher.

Embodiment 34. The computer-implemented method of embodiment 32, wherein the sample is a cell-free DNA (cfDNA) sample, and wherein comparing the first transcription factor activity score to the second transcription factor activity score includes: adjusting the first transcription factor activity score based on a tumor fraction in the sample to create a first normalized transcription factor activity score; adjusting the second transcription factor activity score based on the tumor fraction in the sample to create a second normalized transcription factor activity score; determining a difference between the first normalized transcription factor activity score and the second normalized transcription factor activity score; and comparing an absolute value of the difference to a threshold difference value; and wherein identifying the first phenotype of interest or the second phenotype of interest as the phenotype in the sample based on the comparison includes: in response to determining that the absolute value of the difference is greater than the threshold difference value, identifying the first phenotype of interest or the second phenotype of interest as the phenotype in the sample based on which transcription factor activity score is higher.

Embodiment 35. The computer-implemented method of any one of embodiment 23-34, wherein the first phenotype of interest is a first prostate cancer phenotype, and wherein the second phenotype of interest is a second prostate cancer phenotype.

Embodiment 36. The computer-implemented method of embodiment 35, wherein the first prostate cancer phenotype is an AR expressing phenotype, and wherein the second prostate cancer phenotype is an ASCL1 expressing phenotype.

Embodiment 37. A computer-readable storage medium having computer-executable instructions stored thereon that, in response to execution by one or more processors of a computing system, cause the computing system to perform actions as recited in any one of embodiment 23-36.

Embodiment 38. A computing system configured to perform actions as recited in any one of embodiment 23-36.

The following examples are provided to illustrate certain features and/or embodiments. These examples should not be construed to limit the disclosure to the particular features or embodiments described.

### Example 1

Genomic DNA (gDNA) Extraction Protocol

For genomic DNA extraction from flash frozen LuCaP PCa xenograft tissue, tissue sections from xenografts were cut into small pieces followed by lysis and genomic DNA isolation using the Qiagen DNeasy Blood & Tissue Kit according to the manufacturer's specifications. The DNAs were eluted in 200 ul of UltraPure™ DNase/RNase-Free Distilled Water.

### Example 2

Cell-Free DNA (cfDNA) Isolation

Blood samples were collected from NSG mice bearing subcutaneous PDX tumors at the time of sacrifice. The blood was collected in Sarstedt Micro sample tube K3 EDTA tubes and processed within 4 hours. All blood samples were sequentially double spun, first at 2,500×g for 10 minutes followed by a 16,000×g centrifugation of the plasma fraction for 10 minutes at room temperature. Processed plasma samples were preserved in clean, screw-capped cryo-microfuge tubes and stored at −80° C. prior to cfDNA isolation.

The QIAamp Circulating Nucleic Acid Kit was used to isolate cfDNA from PDX mouse-derived plasma using the recommended protocol. Isolated cfDNA was quantified using the Qubit dsDNA HS assay (Invitrogen) and the cfDNA fragment size profiles were analyzed using TapeStation HS D5000 assay (Agilent).

### Example 3

### EM-Seq DNA Library Preparation and Sequencing

The control DNAs; pUC19 (lilac) and the unmethylated lambda control (lilac) diluted 1:100 using 0.1× TE were fragmented to an average insert size of 240-290 bp and spiked into 50 μl of cfDNA preparation and for gDNA preparation, 50 μl of genomic DNA and spiked control DNA mix is fragmented to an average insert size of 240-290 bp. Fragmentation was done using Covaris instrument in microTUBE-50 AFA Fiber Screw-Cap sonication tubes for 100 secs. For each sample, 10-100 ng of extracted DNA was subjected to EM-seq library preparation using NEBNext Enzymatic Methyl-seq kit (NEB, #E7120S) following the manufacturer's instructions. Briefly, extracted cf/gDNA were mixed with spike-in controls and incubated with 10 μL End Prep Mix at 20° C. for 30 min and followed by 65° C. for 30 min. The DNA then was ligated with methylated adaptors at 20° C. for 15 min, purified with 110 μL magnetic beads, and eluted with 28 μL elution buffer. The purified DNA was used for methylcytosine oxidation with the 17 μL TET2 reaction mix and 5 μL Fe (II) solution and incubated at 37° C. for 1 h. The reaction was stopped by adding 1 μL of Stop Reagent and incubating at 37° C. for 30 min. The oxidated DNA was purified with 90 μL magnetic beads and eluted in 16 μL elution buffer. 4 μL Formamide (Sigma-Aldrich, #F9037-100 ML) was added to denature DNA at 85° C. for 10 min and deamination was immediately carried out by adding 80 μL APOBEC reaction mix to the tube and followed by incubation at 37° C. for 3 h. The treated DNA was then purified with 100 μl magnetic beads. Indexed primers and NEBNext Q5U Master Mix (NEB, #E7120S) were added to purified DNA for 8 cycles of amplification, and each amplified library was purified with 0.9× volume of magnetic beads. EM-seq library profiles were analyzed using TapeStation HS D5000 assay (Agilent) and quantified using the Qubit dsDNA HS assay (Invitrogen).

EM-seq libraries were sequenced on a NovaSeq 6000 sequencer (Illumina) in paired-end 150 bp mode.

### Example 4

### EM-Seq Data Processing

Raw enzymatic methyl-seq (EM-seq) paired-end fastq reads were first trimmed using Trim Galore version 0.6.6 with a 10 nucleotide trim from both the 3′ and 5′ ends of both reads and then aligned to a concatenated reference genome of UCSC hg19, M77789.2 Cloning vector pUC19, and J02459.1 Escherichia phage Lambda using Bismark version 0.23.0. For samples collected from mouse models, UCSC mm10 was also included. Bismark was further used to deduplicate the alignments and extract methylation call files which report the percentage of methylated cytosines and the coverage at each position as well as bedGraph and BigWig files. Samtools was used to extract reads that aligned to each species of the concatenated genome (genome subtraction). Picard toolkit was utilized to collect QC metrics with CollectInsertSizeMetrics, CollectWgsMetrics, and CollectAlignmentSummaryMetrics. IchorCNA was used to estimate copy number and tumor fraction.

### Example 5

### Generate Methylation Profiles.

A custom python script takes a values file as input (bedGraph or BigWig) and computes summary methylation profile at all binding sites listed in a peak file (bed format). The script first uses deepTools version 5.3.1 computeMatrix function for a given window size (+/−1005 bp with 15 bp bins). Next the script takes the output of computeMatrix to generate the summary methylation profile which includes, mean, interquartile range (25%, 50% and 57%) and standard deviation at every bin of +/−1005 window.

### Example 6

### Estimating TFAScores

To estimate fraction of tumor expressing AR and ASCL1, binding sites were first obtained from ChIP-seq performed on prostate cancer cell lines (LNCaP and H660 respectively) and molecular subtype specific Xenograft tissue. ASCL1 is a master neuroendocrine transcription factor expressed in >90% of all NEPCs. Sites of TF binding as measured by peaks in ChIP-seq studies were called using MACS2 a standard software application that determines aggregates of reads in ChIP-seq into discrete mappable peaks with a q-value threshold of 0.1. A custom tool extracts methylation values from value files (BigWig) to compute summary methylation profile at all genome-wide binding sites for given transcription factor binding sites (e.g., AR and ASCL1) listed in a bed format. Metrics such as mean (μ) and variance (σ) of methylation at all given peak sites is used to generate the summary profiles from pure Xenograft cfDNA and healthy cfDNA are used as priors to develop an unsupervised generative mixture model.

P(x1,x2|θ)=α*P(x1|μ1,σ1)+(1−α)*P(x2|μ2,σ1)

Where P(x1, x2|θ) is the joint probability of the two reference biological sample sets with known molecular subtype/TF activity (for example, x1 maybe AR+ and x2 represents the background/healthy sample (for e.g., blood CfDNA or x1 maybe NE+ and x2 is the background/healthy sample) given θ representing the model parameters (μ, σ). For a given sample the model uses this to estimate proportion of molecular phenotype cfDNA in background blood cfDNA. α is the mixture weight which represents the proportions components in the mixture. P(x1|μ1, σ1) and P(x2|μ2, σ1) represents the probability density function of the pure subtype cfDNA and healthy cfDNA, respectively. Pure subtype cfDNA is cfDNA which is derived from subtype specific PDX models and normalized against background blood cfDNA from mouse by aligning to mouse genome. Finally, an Expectation-Maximization (EM) process estimates α by iteratively refining the solution for the above equation. α is considered as the TFAScore for a given sample. TFAScore for each TF (AR and ASCL1) is calculated independently. The final output for a given sample includes AR TFAScore and ASCL1 TFAScore as estimated by the mixture models and tumor fraction (TFx) in cell-free DNA as estimated by ichorCNA.

### Example 7

### Classifying Subtype

To classify subtypes from tissue biopsy, the difference (Δ) of AR TFAScore (α^{AR}) and ASCL1 TFAScore (α^{ASCL1}) was determined. Samples that had difference greater or lesser than a defined threshold (0.05) got classified as AR expressing (i.e., ARPC) or as ASCL1 expressing (i.e., NEPC) respectively.

Δ=α^{AR}−α^{ASCL1}

Similarly, to classify subtypes from cfDNA samples tumor fraction normalized difference (ΔTFx) is used, where difference greater or lesser than a defined threshold (0.05) got classified as AR expressing (i.e., ARPC) or as ASCL1 expressing (i.e., NEPC) respectively. ΔTFx is calculated using AR (α^{AR}) and ASCL1 (a^{ASCL1}) TFAScore and tumor fraction (TFx) from ichorCNA.

$$Δ TFx = \frac{{α}^{AR}}{TFx} - \frac{{α}^{ASCL 1}}{TFx}$$

### Example 8

### TFAScore Performance

i) Methylation Patterns at Transcription Factor Binding Sites are Tightly Linked to Clinically Relevant Molecular Prostate Cancer Subtypes (TFAScore).

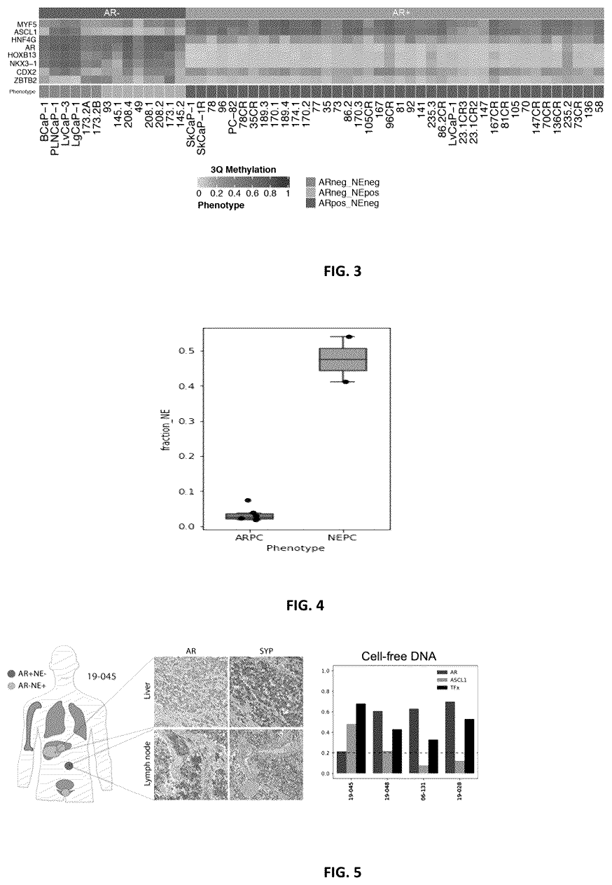

To determine the performance of the methylation based molecular classifier a series of well characterized prostate cancer patient derived xenograft (PDX) models for which molecular phenotypes have been established using orthogonal methods, were first tested. It was observed that TFAScore can accurately distinguish AR+ from AR− (FIG. 2) and NE+ from NE− tumors. Beyond this clinically established subtyping other transcription factors that show distinct binding site methylation alterations in advanced PC (FIG. 3), were also identified. For instance, using the modular analysis framework disclosed herein which allows for the assessment of any TF given ChIP-seq peak files are provided, activity of HNF4G, a gastrointestinal transcription factor, in a subset of cases, was demonstrated. Importantly HNF4G has been previously associated with therapy resistance. These data highlight the broad applicability of the disclosed methods for tumor classicization.
ii) Determining Molecular Phenotype from cfDNA (TFAScore)

From a clinical perspective, the detection of NEPC is of the highest priority. To this end, TFAScore, an algorithm that can detect any evidence of NEPC from cfDNA, was first developed. This model was calibrated with PDX cfDNA and applied to two independent patient cohorts. In both cohorts this approach achieved 100% sensitivity and specificity for the detection of NEPC (FIG. 4).
iii) Quantitative assessment of individual admixed phenotypes

Since metastatic PC often presents as a complex admixture of different molecular phenotypes present in different anatomic sites (FIG. 5), an approach that would allow for a quantitative assessment cfDNA derived from AR+ and NE+ tumors in a given patient, was developed. In benchmarking experiments in patients for whom the entire metastatic tumor burden was comprehensively sampled by a rapid autopsy and cfDNA was analyzed using TFAScore, even small subtype admixtures (reflective of different molecular phenotypes in different metastatic sites) could be accurately deconvoluted and quantified (FIG. 5).

Together these data and studies establish that TFAScore provides a novel quantitative approach and/or a metric for determining molecular subtypes from cfDNA, therefore addressing the critical clinical need for predictive biomarkers that can guide treatment decisions in advanced metastatic PC.

1. A method of treatment of prostate cancer comprising:
(i) determining a molecular phenotype of the prostate cancer, the method comprising:
(a) obtaining a cell-free biological sample from a subject suffering from prostate cancer;
(b) isolating/obtaining total DNA from the cell-free biological sample of the subject;
(c) determining genome-wide methylation profile at a plurality of transcription factor binding sites in the total DNA of the subject suffering from prostate cancer to obtain genome-wide methylation sequence reads at each of the plurality of transcription factor binding sites;
(d) analyzing, including aligning and/or comparing the genome-wide methylation sequence reads obtained in step (c) to genome-wide methylation sequence reads of a reference genome at the plurality of transcription factor binding sites; and
(e) determining alterations in methylation status at the plurality of transcription factor binding sites in the total DNA of the subject suffering from prostate cancer based on the analysis in step (d);
(f) generating a summary methylation profile, wherein the summary methylation profile comprises a pattern of the total DNA that are methylated and/or unmethylated at the plurality of transcription binding sites in the total DNA of the cell-free biological sample; and
(g) generating a metric/quantitative score to determine the molecular phenotype of the prostate cancer; and
(ii) administering to the subject an effective amount of at least one therapeutic agent based on the determination of the molecular phenotype of the prostate cancer.

2. The method of claim 1, wherein the cell-free biological sample comprises blood, plasma, and/or a bodily fluid sample.

3. The method of claim 1, wherein the step of determining genome-wide methylation profile at a plurality of transcription binding sites in the total DNA comprises:
(a) treating the total DNA with a first agent that modifies methylated cytosine residues in the total DNA;
(b) subjecting the total DNA with the modified methylated cytosine to a second agent to convert unmodified cytosines to obtain uracil comprising DNA strands;
(c) amplifying the uracil comprising DNA strands; and
(d) sequencing the amplified DNA to obtain a library.

4. The method of claim 3, wherein the method further comprises obtaining a respective normalized value for the methylation status of each of the plurality of transcription binding sites in the total DNA based on a tumor fraction in the cell-free biological sample of the subject.

5. The method of claim 3, wherein the methylated cytosine residues comprise 5 methyl cytosine and 5-hydroxymethyl cytosine.

6. The method of claim 5, wherein the first agent oxidizes the methylated cytosine residues to obtain the modified methylated cytosine residues.

7. The method of claim 3, wherein the second agent is a deamination agent.

8. The method of claim 1, wherein the reference genome comprises a reference genome-wide methylation profile obtained from at least one sample obtained from a subject known to have the molecular phenotype of the prostate cancer.

9. The method of claim 1, wherein the reference genome comprises a reference genome-wide methylation profile obtained from at least one sample obtained from a healthy subject.

10. The method of claim 1, wherein the prostate cancer is a Metastatic castration-resistant prostate cancer.

11. The method of claim 10, wherein the prostate cancer lacks androgen receptor and is characterized by gain of stem-like and neuroendocrine features.

12. The method of claim 1, wherein generating the metric/quantitative score comprises generating a Transcription Factor Activity Score (TFAScore), and wherein the TFAScore is indicative of genome-wide activity of transcription factors in the cell-free biological sample.

13. The method of claim 1, wherein the therapeutic agent is a chemotherapeutic agent.

14. The method of claim 13, wherein the chemotherapeutic agent is selected from a) an anti-hormone treatment; b) a cytotoxic agent; c) a biologic, preferably an antibody and/or a vaccine; and d) a targeted therapeutic agent.

15. A method comprising:
(a) obtaining total DNA from a cell-free biological sample;
(b) modifying the total DNA and amplifying the modified total DNA to obtain a genome-wide methylation profile at a plurality of transcription binding sites in the total DNA;
(c) analyzing, including aligning and/or comparing the genome-wide methylation profile obtained in step (b) to a reference genome-wide methylation profile at the plurality of transcription binding sites in total DNA obtained from a cell-free reference sample;
(d) determining alteration in methylation at the plurality of transcription binding sites based on the analysis in step (c);
(e) generating a summary methylation profile for each of the plurality of transcription binding sites, wherein the summary methylation profile comprises a pattern of the total DNA that are methylated and/or unmethylated at the plurality of transcription binding sites in the total DNA of the cell-free biological sample; and
(f) generating a metric/quantitative score,
wherein the metric/quantitative score comprises generating a Transcription Factor Activity Score (TFAScore).

16. The method of claim 15, wherein the cell-free biological sample is selected from blood, plasma, and a bodily fluid obtained from a subject suffering from prostate cancer.

17. The method of claim 15, wherein the reference genome-wide methylation profile comprises genome-wide methylation profile at the plurality of transcription binding sites in DNA obtained from at least one biological sample of a subject known to have a specific type of prostate cancer.

18. The method of claim 15, wherein the reference genome-wide methylation profile comprises genome-wide methylation profile at the plurality of transcription binding sites in DNA obtained from at least one biological sample of a healthy subject.

19. The method of claim 17, wherein the prostate cancer is a Metastatic castration-resistant prostate cancer.

20. The method of claim 17, wherein the prostate cancer lacks androgen receptor and is characterized by gain of stem-like and neuroendocrine features.

21. The method of claim 15, wherein the TFAScore is indicative of genome-wide activity of transcription factors in the cell-free biological sample.

22. A method of treatment of prostate cancer, the method comprising:
(i) determining a type of prostate cancer by analyzing total DNA obtained from a cell-free biological sample of a subject, wherein the subject is a human, the method comprising:
(a) performing methylation-aware genome-wide sequencing for the total DNA to generate genome-wide sequence reads, wherein the genome-wide sequence reads comprise methylation status at a plurality of transcription binding sites of the total DNA;
(b) analyzing, including aligning and/or comparing to a reference human genome the genome-wide sequence reads obtained in step (a) to identify each of a plurality of transcription binding sites, and using the analysis to obtain methylation status for each of the plurality of transcription binding sites in the total DNA;
(c) generating a summary methylation profile from the methylation status for each of the plurality of transcription binding sites obtained in step (b), wherein the summary methylation profile comprises a pattern of the total DNA that are methylated and/or unmethylated at the plurality of transcription binding sites in the total DNA obtained from the cell-fee biological sample of the subject; and
(d) determining the subject has a specific type of prostate cancer based at least in part on the summary methylation profile, wherein the step of determining the type of prostate cancer comprises analyzing differences between values of the summary methylation profile obtained in step (c) and values of one or more reference methylation profiles, and wherein at least one of the one or more reference methylation profiles is obtained from DNA of at least one biological sample obtained from a subject known to have the specific type of prostate cancer and/or a healthy subject;
(ii) selecting at least one therapeutic modality based on the type of prostate cancer; and
(iii) administering to the subject an effective amount of the at least one therapeutic modality.

23. A computer-implemented method of determining a phenotype in a sample from a subject, the method comprising:
receiving, by a computing system, sequencing data for the sample;
aligning, by the computing system, the sequencing data to a reference genome to generate alignment data;
processing, by the computing system, the alignment data to create methylation data;
processing, by the computing system, the methylation data to create summary methylation data for transcription factor binding sites associated with a first phenotype of interest and a second phenotype of interest;
determining, by the computing system, a first transcription factor activity score for the first phenotype of interest and a second transcription factor activity score for the second phenotype of interest based on the summary methylation data; and
determining, by the computing system, the phenotype in the sample based on the first transcription factor activity score and the second transcription factor activity score.

24. The computer-implemented method of claim 23, wherein determining the first transcription factor activity score includes determining a mixture weight that represents a proportion of the first phenotype of interest and a proportion of a sample background in the sample; wherein the mixture weight is used as the first transcription factor activity score.

25. The computer-implemented method of claim 24, wherein determining the mixture weight includes determining a value for a in:
P(x₁,x₂|θ)=α*P(x₁|μ₁,σ₁)+(1−α)*P(x₂|μ₂,σ₁)
wherein α is the mixture weight;
wherein P(x₁|μ₁, σ₁) is a probability density function for the first phenotype of interest given a mean of the first phenotype of interest and a standard deviation of the first phenotype of interest determined from a known sample;
wherein P(x₂|μ₂, σ₁) is a probability density function for the sample background given a mean of the sample background and the standard deviation of the first phenotype of interest; and
wherein P(x₁, x₂|θ) is a joint probability density function for the first phenotype of interest and the sample background given features based on the summary methylation data.

26. The computer-implemented method of claim 25, wherein the features based on the summary methylation data include a mean and a standard deviation.

27. The computer-implemented method of claim 24, wherein determining the mixture weight includes estimating the mixture weight using an Expectation-Maximization process.

28. The computer-implemented method of claim 23, wherein processing the methylation data to create summary methylation data for transcription factor binding sites associated with the first phenotype of interest and the second phenotype of interest includes: extracting methylation values for windows around the transcription factor binding sites associated with the first phenotype of interest and the second phenotype of interest; and; calculating summary metrics for the extracted methylation values.

29. The computer-implemented method of claim 28, wherein the summary metrics include one or more of a mean, an interquartile range, or a standard deviation.

30. The computer-implemented method of claim 28, wherein the summary metrics are calculated at a plurality of bins within each window.

31. The computer-implemented method of claim 30, wherein a size of each bin of the plurality of bins is 15 bp.

32. The computer-implemented method of claim 23, wherein determining the phenotype in the sample based on the first transcription factor activity score and the second transcription factor activity score includes:
comparing the first transcription factor activity score to the second transcription factor activity score; and
identifying the first phenotype of interest or the second phenotype of interest as the phenotype in the sample based on the comparison.

33. The computer-implemented method of claim 32, wherein the sample is from a tissue biopsy, and wherein comparing the first transcription factor activity score to the second transcription factor activity score includes:
determining a difference between the first transcription factor activity score and the second transcription factor activity score; and
comparing an absolute value of the difference to a threshold difference value; and
wherein identifying the first phenotype of interest or the second phenotype of interest as the phenotype in the sample based on the comparison includes:
in response to determining that the absolute value of the difference is greater than the threshold difference value, identifying the first phenotype of interest or the second phenotype of interest as the phenotype in the sample based on which transcription factor activity score is higher.

34. The computer-implemented method of claim 32, wherein the sample is a cell-free DNA (cfDNA) sample, and wherein comparing the first transcription factor activity score to the second transcription factor activity score includes:
adjusting the first transcription factor activity score based on a tumor fraction in the sample to create a first normalized transcription factor activity score;
adjusting the second transcription factor activity score based on the tumor fraction in the sample to create a second normalized transcription factor activity score;
determining a difference between the first normalized transcription factor activity score and the second normalized transcription factor activity score; and
comparing an absolute value of the difference to a threshold difference value; and
wherein identifying the first phenotype of interest or the second phenotype of interest as the phenotype in the sample based on the comparison includes:
in response to determining that the absolute value of the difference is greater than the threshold difference value, identifying the first phenotype of interest or the second phenotype of interest as the phenotype in the sample based on which transcription factor activity score is higher.

35. The computer-implemented method of claim 23, wherein the first phenotype of interest is a first prostate cancer phenotype, and wherein the second phenotype of interest is a second prostate cancer phenotype.

36. The computer-implemented method of claim 35, wherein the first prostate cancer phenotype is an AR expressing phenotype, and wherein the second prostate cancer phenotype is an ASCL1 expressing phenotype.

37. A computer-readable storage medium having computer-executable instructions stored thereon that, in response to execution by one or more processors of a computing system, cause the computing system to perform actions as recited in claim 23.

38. A computing system configured to perform actions as recited in claim 23.
